(19) United States
(12) Patent Application Publication
(10) Pub. No.: US 2021/0118563 A1
(43) Pub. Date: Apr. 22, 2021

(54) Method For Providing Virtual Triage

(71) Applicants: Mary Reaston, Carlsbad, CA (US); Avrohom Light, Los Angeles, CA (US)

(72) Inventors: Mary Reaston, Carlsbad, CA (US); Avrohom Light, Los Angeles, CA (US)

(21) Appl. No.: 16/658,114

(22) Filed: Oct. 20, 2019

(51) Int. Cl. G16H 40/67; G16H 50/20; G16H 50/30; G16H 80/00

(52) U.S. Cl. CPC G16H 40/67; G16H 80/00; G16H 50/30; G16H 50/20

(57) ABSTRACT

A method for providing virtual triage (MPVT) and a virtual clinic with diagnosis and treatment that preferably functions in combination with an Electrodiagnostic Functional Assessment (EFA) device, and that can be used for a person in general or particularly for an employee of a company who experiences an on-the-job medical issue or injury. The steps are typically the first action taken after person or employee experiences a medical issue or injury, and the steps are collectively known as Physicians Triage. The MPVT steps facilitate the quick and accurate diagnosis of an issue of injury and the determination of action to be taken such as immediate or postponed treatment. The MPVT can also be utilized with information that is used as baseline data for comparison/verification of an employee medical issue or injury that occurred at a later date. The baseline data can establish if an issue or injury such as a reported on-the-job injury is a new/current injury or a pre-existing condition.

### TECHNICAL FIELD

The invention generally pertains to medical procedures, and more particularly to method for providing virtual triage particularly for employee on-the-job complaints or injuries that are remotely or non-remotely determined and that maybe based on previous baseline test results acquired from an electrodiagnostic functional assessment device.

### BACKGROUND ART

Medical issues including medical complaints or injuries are one of the most prevalent problems affecting people throughout the world. A medical issue can happen to any person and at any time from relaxing at home or when at work. When a person does experience a medical issue, typically the first step in addressing the issue is to perform what is known as triage, during which a medical professional such as a nurse or physician will acquire information about the person, the medical issue and will perform an initial medical evaluation.

During triage different levels of condition are often assigned colors to identify the requirement for treatment. For example, red indicates a need for immediate resuscitation within one minute, orange indicates a very urgent need within ten minutes, yellow indicates an urgent need within sixty minutes, green indicates a standard/general need within one hundred and twenty minutes, and blue indicates a non-urgent need that should be threated within two hundred and forty minutes (or more). While these methods can be effective, especially in a controlled environment, with a limited number of patients, during major emergency situations when a large number of patients require treatment it can be difficult to manage the need for treatment and assign colors. This is particularly problematic when multiple doctors and medical personnel are assigning and prioritizing by color. Additionally, triage is typically performed quickly, often under difficult conditions, and therefore there is chance of error when a patient is being examined and treatment recommended. Another problem with current triage is that a patient's medical record/history is often not available, or a doctor or medical personnel do not have the time and ability to review a patient's entire medical history, which often shows existing or underlying issues that could affect recommended treatment.

Triage is commonly performed at a medical facility where a person has gone for treatment, but triage can be performed at any location or by telephone or video conference. The more quickly triage is performed facilitates more effective treatment of the medical issue. If a medical issue occurs at a remote location, quick triage at the location can provide medical personnel with important information that is needed to best treat an injured person.

Virtual triage is an on-line version of triage, and can be used in combination with a virtual clinic. As with conventional triage, virtual triage and a virtual clinic can be accomplished by performing a basic examination and asking questions. Additionally a device such as an electrodiagnostic functional assessment (EFA) device can be used for the virtual clinic, and the virtual triage can provide a bookend solution program. An EFA can perform tests such as range-of-motion (ROM), functional capacity evaluation (FCE) and others which produce a more accurate and complete assessment of a complaint or injury An EFA can also be used at remote locations and the information acquired can be sent to a medical facility before an injured person arrives at the facility. This capability is very important especially for on-the-job injuries that occur at a person's work location.

On-the-job complaints and injuries are particularly concerning to employers and insurance companies who often must pay significant costs when one of their employees is injured on-the-job. Also an EFA can be used to acquire baseline data for each employee. The baseline data can be stored and then if an employee asserts that an on-the-job complaint or injury has occurred, the baseline data will prove whether the complaint or injury is new or was a pre-existing condition. This would greatly reduce the occurrence of workers compensation fraud.

What is needed is a method of performing a virtual triage, preferably a short time after a medical complaint is made or an injury occurs (or is alleged to have occurred). The virtual triage will optimally be used with an EFA baseline program. This method would be a more efficient way of quickly performing triage, even at a remote location such as a jobsite. A determination could be made if a person with a complaint or injury requires immediate medical care of if medical care can be postponed, or to see if the person who has had an EFA baseline can have a second EFA test. Also, by comparing baseline data with current virtual triage acquired information, a person/employee complaint or injury can be established as new or pre-existing.

Also, current physician triage methods recommend treatment prior to an EFA post-loss test, which would possibly create a worker's compensation claim. By using a virtual triage in which evidence is workflow based, individuals can be referred to an EFA to determine if there was an actual injury or change in condition.

A search of the prior art did not disclose any literature or patent that read directly on the claims of the instant invention. However, the following U.S. patents are considered related:

| PATENT NO. | INVENTOR | ISSUED |
|---|---|---|
| 8,630,867 | Yoo | Jan. 14, 2014 |
| 2002/0065682 | Goldenberg | May 30, 2002 |
| 2010/00222649 | Schenberg | Sep. 2, 2010 |
| 2016/0117451 | Reaston, et al | Jan. 4, 2016 |
| 2016/0147957 | Reaston, et al | Nov. 24, 2016 |

The U.S. Pat. No. 8,630,867 patent discloses a remote medical-diagnosis system performing biological, chemical or biochemical reactions with a sample, and having a barcode or a RF IC: bioanalytical device analyses results of reactions and includes a reader to authenticate the measured data. A virtual doctor provides a user with guidelines and instructions as how to use the device, and provides the user with a consulting service. A diagnosis unit self-analyzes the measured data and outputs results of a diagnosis. A terminal provides the user with a consulting service from a medical expert or a virtual doctor and a remote diagnosis server connects the user with the medical expert.

The 2002/0065682 publication discloses an interactive network-based health information system that provides up-to-date medical information directly to a user. The user can issue specific follow-up questions, initiate a discussion with a professional, and establish a doctor-person relationship. The system provides for remote monitoring and diagnosis of the person and for remote treatment.

The 2010/00222649 publication discloses remote medical servicing between a consumer and a medical service provider. The invention includes a request from a person to consult with a medical service provider having a service provider profile. A medical service provider satisfying attributes is identified; a communication channel; real-time communication between the person and the medical service provider and a measurement of a physiological parameter of the person is received over the communication channel.

The 2016/0117451 publication discloses a method for determining change in condition for a soft tissue injury. The method determines if an injury occurs as a result of the course and scope of an individual's employment requirements or activities. The method is designed to maintain objectivity in pre-loss and post-loss injury assessments by utilizing a non-loaning and non-invasive functional assessment that is facilitated by proprietary software.

The 2016/0147957 publication discloses a system and method for a healthcare provider that facilitates the creation of baseline test data and post-loss test data to determine if an individual has experienced a legitimate work-related injury. Additionally, the healthcare provider employs or retains the services of healthcare professionals who provide general injury assessment. The healthcare provider provides a method diagnostic unit, which is known as an EFA, that utilizes electromyography (EMG), range-of-motion (ROM) and functional capacity assessment (FCA). The EFA receives data that pertains to an individual via a real-time communication protocol, or the individual's data is collected and stored on an electronic storage device.

For background purposes and indicative of the art to which the invention relates, reference may be made to the following remaining patents found in the patent search.

| PATENT NO. | INVENTOR | ISSUED |
|---|---|---|
| 7,505,614 | DeLaTorre-Bueno | Mar. 17, 2004 |
| 7,668,733 | Glimp, et al | Feb. 23, 2010 |
| 8,535,224 | Reaston, et al | Sep. 17, 2013 |
| 2006/0161457 | Rapaport, et al | Jul. 20, 2006 |
| 2016/0092646 | Rinkacs, et al | Mar. 31, 2016 |
| 2017/0293730 | Fish, et al | Oct. 12, 2017 |

### DISCLOSURE OF THE INVENTION

A method for providing virtual triage (MPVT) that preferably utilizes an electrodiagnostic functional assessment (EFA) device, and that can be used on persons in general or particularly employees of a company. The MPVT, also known as Physician Triage, is facilitated by performing a series of steps. Since these steps are typically the first action when a person or employee has a medical issue, the initial steps are indicated by and referred to as triage.

The MPVT is preferably utilized in combination with an EFA baseline test. The ability to triage is extremely important for both employers and insurance companies as well as employees who can ensure that better care for work related injuries is provided.

While there is only a single method, there are multiple series of steps with slight variations, which are optimise for when the medical complaint or injury is from person in general or an employee of a company, and whether a baseline test was previously performed and the results are available.

As previously disclosed, the MPVT is particularly effective when used in combination with an EFA baseline test to better address and manage work related injuries.

An example of basic MPVT steps which can be used for a person in general or an employee of a company who has previously had an EFA baseline test, and the person's employee's medical issue or injury is within the scope of the EFA baseline test, are: a person/employee experiences a medical issue or injury,
person/employee undergoes triage and requires a physician triage evaluation,
a case coordinator performs person/employee intake process comprising:
   acquiring person/employee name, address and personal information,
   acquiring person/employee medical history, medical records and primary medical complaint/issue,
   acquiring employer name and address (if applicable),
case coordinator remotely connects person/employee to physician who confirms reason for connection and determines if remote triage should continue or person/employee requires in-person treatment,
physician informs person/employee that second treating physician will contact person/employee in a period of time, if the person/employee outside the EFA program and chooses to visit a virtual clinic,
treating physician contacts person/employee, performs evaluation and recommends treatment,
follow-up appointment for person/employee is scheduled with follow-up physician if required by person's condition,
follow-up physician evaluates if person/employee has shown no improvement during time from scheduling follow-up appointment to the follow-up appointment, and determines if a telemedicine assessment is to be performed to ascertain site-specific treatment, and assessment.

In view of the above disclosure the primary object of the invention is to produce a method for providing virtual triage that can quickly and easily determine the nature and extent of a medical complaint or injury preferably by use of an EFA.

In addition to the primary object, it is also an object of the invention to provide a method for produce a method for providing virtual triage that:
can be used for many types of medical complaints or injuries including soft tissue injuries or non-soft tissue injuries,
utilizes an EFA baseline program to provide medical diagnosis and treatment via a virtual clinic,
is easy to learn and perform,
can be used by various medical professionals,
can provide data that proves if a person or employee is actually experiencing an issue or pain,
utilize triage to better manage an EFA baseline program that determines if a complaint or injury is new or pre-existing,
can be used at a location remote,
can be used with communication means such as a telephone,
is cost effective particularly for employers and insurance companies.

These and other objects and advantages of the present invention will become apparent from the subsequent detailed description of the preferred embodiment and the appended claims taken in conjunction with the accompanying drawings.

### BRIEF DESCRIPTION OF THE DRAWINGS

FIG. 1 is a flow diagram of the steps performed during a method for providing virtual triage (MPVT) for a person in general.

FIG. 2 is a flow diagram of the steps performed during the MPVT for an employee of a company.

FIG. 3 is a flow diagram of first alternate steps performed during the MPVT for an employee of a company.

FIG. 4 is a flow diagram of second alternate steps performed during the MPVT for an employee of a company and when a baseline test has been previously performed.

### BEST MODE FOR CARRYING OUT THE INVENTION

The best mode for carrying out the invention is presented in terms and steps that disclose a preferred embodiment with variations of a method for providing virtual triage (MPVT) 10. Many medical issues such as medical complaints or injuries occur “on the job” to an employee of a company. These medical issues can result in a significant cost for an employer and/or insurance company as well as costly and ineffective treatment for the employee.

When a person such as an employee of a company has a medical issue the initial treatment step is to perform triage. A medical professional such as a nurse or a physician (when necessary) valuates the person to determine the nature and extent of the medical issue. The triage is extremely important and has a major role in determining what actions are required to address the person's medical issue. The greater amount of information that is acquired during triage will make the treatment much better and efficient.

Depending on the information acquired during triage, a physician will determine the subsequent actions taken for the person with the medical issue. These actions can include additional or continuing triage, self-help, therapy or in-person treatment at a medical facility. In the past, physicians and other medical professionals performed triage to simply acquire a basic comprehension of the medical situation they were dealing with. Today there are options that greatly improve medical functions in acquiring information during triage. One such device is a proprietary electrodiagnostic functions assessment (EFA) device which was developed and patented by the applicant of this invention. The EFA can perform multiple tests including range-of-motion (ROM), functional capacity evaluation (FCE) as well as others. These test are extremely important especially when diagnosing and treating medical issues such as musculoskeletal disorder (MSD); chronic pain; headache; temporomandibular joint disorder (TMJ); neck, back and shoulder pain; soft tissue injury, carpal tunnel syndrome, arm or leg pain, and repetitive stress injuries.

The EFA is especially effective for acquiring baseline medical data, can determine whether a medical issue or injury has recently occurred or if the issue/injury is pre-existing. Also, current physician triage methods recommend treatment prior to an EFA post-loss test, which would possibly create a worker's compensation claim. By using a virtual triage in which evidence is workflow based, individuals can be referred to an EFA to determine if there was an actual injury or change in condition. The EFA can also help a physician determine if a medical issue is minor enough to require treatment at an occupational medical clinic or urgent care, or is serious enough to need treatment at an emergency room. Also, the EFA without baseline test results can be utilized with the virtual clinic

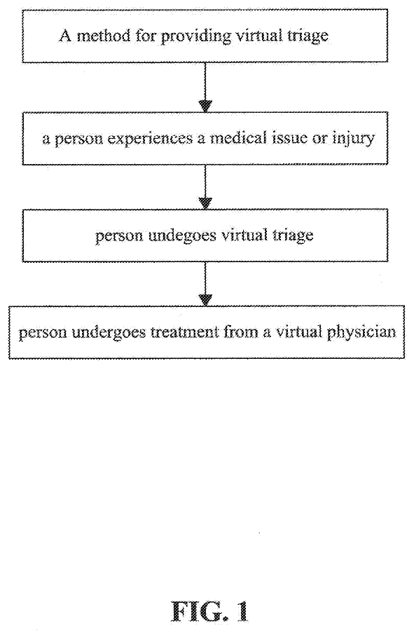

As previously disclosed the MPVT 10 can be effectively utilized for both persons with medical issues or for employees of a company who experience an injury which typically occurs on-the-job. The most basic MPVT steps are shown in FIG. 1 and are comprised of:
a person experiences a medical issue or injury,
person undergoes virtual triage, and
person undergoes treatment from a virtual physician.

In addition to the basic steps, the MPVT steps can include:
the person requesting to see a physician after triage,
person requiring in-person treatment, person is referred to a facility and physician
specifies condition needing further evaluation or treatment, and
scheduling a follow-up appointment within three to seven days.

Further, the triage can determine that the person undergo a second test when the person has conditions within the scope of the MPVT.

After the baseline test, a physician schedules a subsequent test that produces results which are compared of the baseline test results.

Finally, the person can be directed to see a virtual physician after virtual triage, and when the person requires in-person treatment, the person is referred to a facility and physician specifies a medical issue needing further evaluation or treatment which can also be provided by a virtual physician and the treatment facility is in a network of associated facilities.

If a telemedicine assessment is required, the assessment can be performed with the EFA.

While the steps can be utilized for a general person, the steps could also be used for an employee of a company.

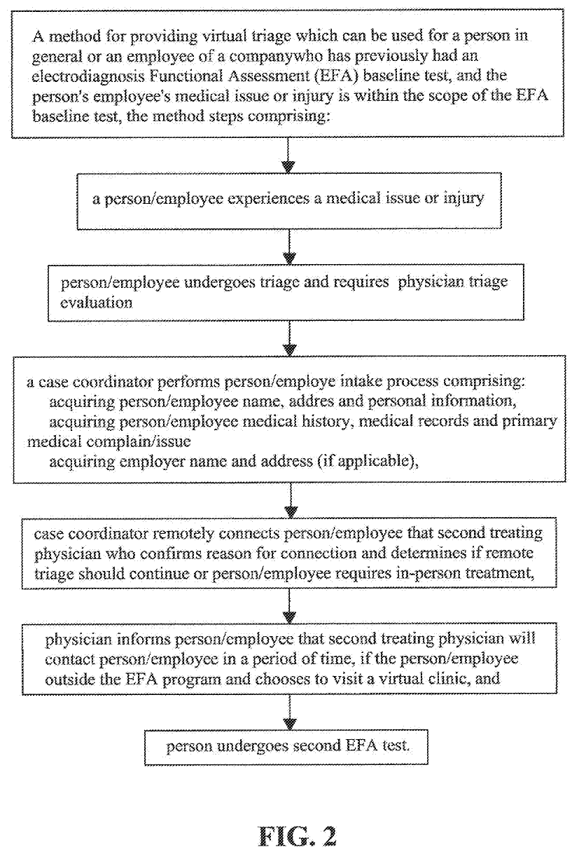

Additional MPVT steps which can be used for a person in general or an employee of a company who has previously had an Electrodiagnosis Functional Assessment (EFA) baseline test, and the person's employee's medical issue or injury is within the scope of the EFA baseline test, are shown in FIG. 2 and comprised of:
a person/employee experiences a medical issue or injury,
person/employee undergoes triage and requires a physician triage evaluation,
a case coordinator performs person/employee intake process comprising:
   acquiring person/employee name, address and personal information,
   acquiring person/employee medical history, medical records and primary medical complaint/issue,
   acquiring employer name and address (if applicable),
case coordinator remotely connects person/employee to physician who confirms reason for connection and determines if remote triage should continue or person/employee requires in-person treatment,
physician informs person/employee that second treating physician will contact person/employee in a period of time, if the person/employee outside the EFA program and chooses to visit a virtual clinic, and
person undergoes second EFA test.

When there are no baseline test results and the employee insists on seeing a physician during virtual triage, physician and case coordinator refer employee to an occupational medical clinic, urgent care or emergency room approved by employer, and physician forwards medical history and instructions from virtual triage to occupational medical clinic, urgent care or emergency room thereby allowing the employee to be treated for specific complaint or injury.

When an employee requires in-person treatment, or when an employee insists on seeing a physician during virtual triage, the physician and a medical professional such as a case coordinator will refer the employee to an appropriate employer approved facility such as an occupational medical clinic, urgent care or emergency room.

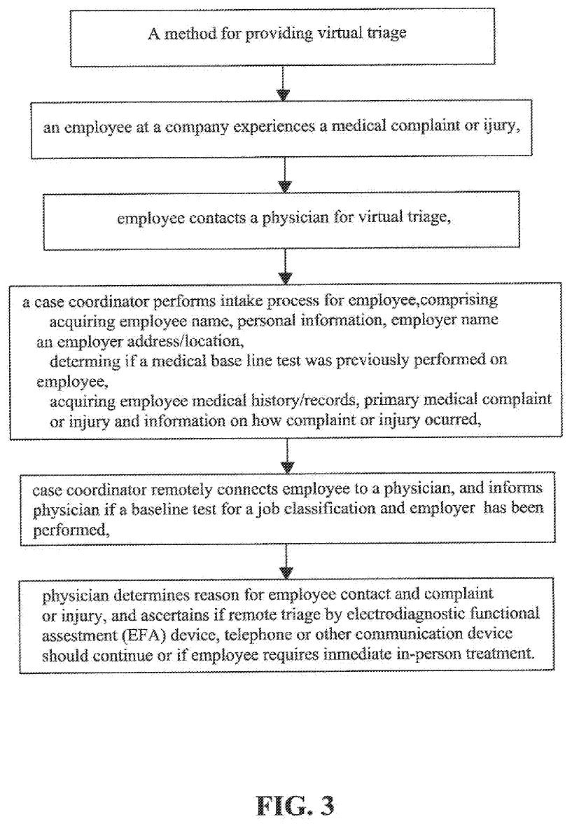

First alternate MPVT steps are shown in FIG. 3 and are comprised of:
an employee at a company experiences a medical complaint or injury,
employee contacts a physician for triage,
   a case coordinator performs intake process for employee, comprising:
acquiring employee name, address, personal information, employer name and employer address/location,
determining if a medical baseline test was previously performed on employee,
acquiring employee medical history/records, primary medical complaint or injury and information on how compliant or injury occurred,

case coordinator remotely connects employee to a physician, and informs physician if a baseline test for a job classification and employer has been performed,

physician determines reason for employee contact and complaint or injury, and ascertains if remote triage by electrodiagnostic functional assessment (EFA) device, telephone or other communication device should continue or if employee requires immediate in-person treatment.

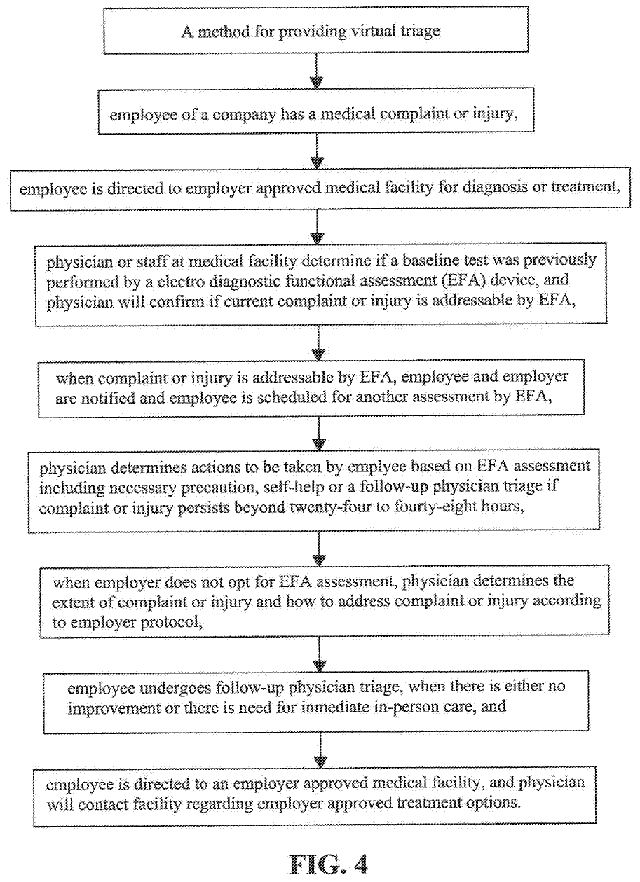

Second alternate MPVT steps are shown in FIG. 4 and are comprised of:
employee of a company has a medical complaint or injury,
employee is directed to employer approved medical facility for diagnosis or treatment,
physician or staff at medical facility determine if a baseline test was previously performed by a electro diagnostic functional assessment (EFA) device, and physician will confirm if current complaint or injury is addressable by EFA,
when complaint or injury is addressable by EFA, employee and employer are notified and employee is scheduled for another assessment by EFA,
physician determines actions to be taken by employee based on EFA assessment including necessary precautions, self-help or a follow-up physician triage if complaint or injury persists beyond twenty-four to forty-eight hours,
when employer does not opt for EFA assessment, physician determines the extent of complaint or injury and how to address complaint or injury according to employer protocol,
employee undergoes follow-up physician triage, when there is either no improvement or there is need for immediate in-person care, and
employee is directed to an employer approved medical facility, and physician will contact facility regarding employer approved treatment options.

For any of the MPVT steps,

If an employee insists on seeing an in-person physician during initial virtual triage, the physician in coordination with the care coordinator will refer employee preferably to an occupational medical clinic approved by the employer. The care coordinator will also forward instructions and medical records to the medical clinic where the employee is to be seen for a specific complaint or injury only and no other condition.

Also, during the MPVT steps a physician will not prescribe medication to employee or recommend they seek medical treatment unless employee requires immediate attention and meets triage protocol specific to each employer. Physician will not recommend employee take any time off. Either the employee will be referred to an occupational medical facility or appropriate facility, or the employee will be advised the medical issue or injury does not require immediate medical attention.

While the invention has been described in detail and pictorially shown in the accompany drawing it is not to be limited to such details, since many changes and modification maybe made to the invention without departing from the spirit and the scope thereof. Hence, it is described to cover any and all modifications and forms which may come within the language and scope of the claims.

1. A method for providing virtual triage (MPVT) comprising the following steps:
a person experiences a medical issue or injury,
person undergoes virtual triage, and
person undergoes treatment from a virtual physician.

2. The method for providing virtual triage of claim 1, further comprising a step of the person requesting to see a physician after triage.

3. The method for providing virtual triage of claim 1, wherein when person requires in-person treatment, person is referred to a facility and physician specifies condition needing further evaluation or treatment.

4. The method for providing virtual triage of claim 1, further comprising scheduling a follow-up appointment.

5. The method for providing virtual triage of claim 4, wherein the follow-up appointment is scheduled within three to seven days.

6. The method for providing virtual triage of claim 1, wherein the assessment is performed with an Electrodiagnostic Functional Assessment (EFA) device.

7. The method for providing virtual triage of claim 1, further comprising the triage determining that the person undergo a second test when the person has conditions within the scope of the MPVT.

8. The method for providing virtual triage of claim 1, wherein after the baseline test, a physician schedules a subsequent test that produces results which are compared to the baseline test results.

9. The method for providing virtual triage of claim 1, further comprising the person directed to see a virtual physician after virtual triage.

10. The method for providing virtual triage of claim 1, wherein when the person requires in-person, treatment, the person is referred to a facility and physician specifies a medical issue needing further evaluation or treatment.

11. The method for providing virtual triage of claim 1, wherein when the person requires treatment, the treatment is provided by a virtual physician.

12. The method for providing virtual triage of claim 1, wherein when person requires treatment, the person will undergo an electrodiagnostic functional assessment from the EFA device.

13. The method for providing virtual triage of claim 1, wherein the treatment facility is included in a network of associated facilities.

14. The method for providing virtual triage of claim 1, wherein a telemedicine assessment is performed with an Electrodiagnosis Functional Assessment (EFA) device.

15. A method for providing virtual triage (MPVT) which can be used for a person in general or an employee of a company who has previously had an Electrodiagnosis Functional Assessment (EFA) baseline test, and the person's employee's medical issue or injury is within the scope of the EFA baseline test, the method steps comprising:
a person/employee experiences a medical issue or injury,
person/employee undergoes triage and requires a physician triage evaluation,
a case coordinator performs person/employee intake process comprising:
acquiring person/employee name, address and personal information,
acquiring person/employee medical history, medical records and primary medical complaint/issue,
acquiring employer name and address (if applicable),
case coordinator remotely connects person/employee to physician who confirms reason for connection and determines if remote triage should continue or person/employee requires in-person treatment,
physician informs person/employee that second treating physician will contact person/employee in a period of time, if the person/employee outside the EFA program and chooses to visit a virtual clinic, and
person undergoes second EFA test.

16. A method for providing virtual triage comprising the following steps:
an employee at a company experiences a medical complaint or injury,
employee contacts a physician for triage,
a case coordinator performs intake process for employee, comprising:
acquiring employee name, address, personal information, employer name and employer address/location,
determining if a medical baseline test was previously performed on employee,
acquiring employee medical history/records, primary medical complaint or injury and information on how compliant or injury occurred,
case coordinator remotely connects employee to a physician, and informs physician if a baseline test for a job classification and employer has been performed,
physician determines reason for employee contact and complaint or injury, and ascertains if remote triage by electrodiagnostic functional assessment (EFA) device, telephone or other communication device should continue or if employee requires immediate in-person treatment.

17. The method for providing virtual triage of claim 16, wherein when employee requires in-person treatment after triage, employee is referred to an employer approved occupational medical clinic, urgent care or emergency room, and physician will follow-up with facility on results.

18. The method for providing virtual triage of claim 16, wherein when employee insists on seeing a physician during triage, physician and case coordinator refer employee to an occupational medical clinic, urgent care or emergency room approved by employer, and physician forwards medical history and instructions from triage to occupational medical clinic, urgent care or emergency room thereby allowing the employee to be treated for specific complaint or injury.

19. A method for providing virtual triage comprising the following steps:
employee of a company has a medical complaint or injury,
employee is directed to employer approved medical facility for diagnosis or treatment,
physician or staff at medical facility determine if a baseline test was previously performed by a electro diagnostic functional assessment (EFA) device, and physician will confirm if current complaint or injury is addressable by EFA,
when complaint or injury is addressable by EFA, employee and employer are notified and employee is scheduled for another assessment by EFA,
physician determines actions to be taken by employee based on EFA assessment including necessary precautions, self-help or a follow-up physician triage if complaint or injury persists beyond twenty-four to forty-eight hours,
when employer does not opt for EFA assessment, physician determines the extent of complaint or injury and how to address complaint or injury according to employer protocol,
employee undergoes follow-up physician triage, when there is either no improvement or there is need for immediate in-person care, and
employee is directed to an employer approved medical facility, and physician will contact facility regarding employer approved treatment options.

20. The method for providing virtual triage of claim 19, wherein the medical issue is selected from the group consisting of musculoskeletal disorder (MSD), chronic pain; headache; temporomandibular joint disorder (TMJ); neck, back and shoulder pain, carpal tunnel syndrome, arm or leg pain and repetitive stress injuries.
